(19) United States
(12) Patent Application Publication
(10) Pub. No.: US 2012/0028976 A1
(43) Pub. Date: Feb. 2, 2012

(54) PHARMACOKINETICALLY-BASED DOSING REGIMENTS OF A THROMBIN RECEPTOR ANTAGONIST

(76) Inventors: Larisa Reyderman, Watchung, NJ (US); Teddy Kosoglou, Jamison, PA (US); Enrico P. Veltri, Princeton, NJ (US)

(21) Appl. No.: 13/129,725

(22) PCT Filed: Nov. 16, 2009
(86) PCT No.: PCT/US09/64503

§ 371 Date: Oct. 11, 2011

Related U.S. Application Data
(60) Provisional application No. 61/115,290, filed on Nov. 17, 2008.

(51) Int. Cl. A61K 31/443; A61K 31/5377; A61P 9/00; A61K 31/444

(52) U.S. Cl. 514/235.2; 514/333; 514/337

(57) ABSTRACT

Dosing regimens based on the pharmacokinetic characteristics of a thrombin receptor antagonist are disclosed. In some embodiments, the dosing regimens result in mean plasma concentrations. Also disclosed are methods of treating acute coronary syndrome and peripheral arterial disease, and of effecting secondary prevention, by orally administering thrombin receptor antagonists according to such dosing regimens.

### FIELD OF THE INVENTION

The invention relates to pharmaceutical compositions and dosing regimens for delivery of a thrombin receptor antagonist.

### BACKGROUND

Thrombin is known to have a variety of activities in different cell types and thrombin receptors are known to be present in such cell types as human platelets, vascular smooth muscle cells, endothelial cells and fibroblasts. It is therefore possible that thrombin receptor antagonists, also known as protease activated receptor (PAR) antagonists, will be useful in the treatment of thrombotic, inflammatory, atherosclerotic and fibroproliferative disorders, as well as other disorders in which thrombin and its receptor play a pathological role.

U.S. Pat. No. 7,304,078 discloses a genus of compounds, including a specific thrombin receptor antagonist compound identified as Example 2, herein identified as COMPOUND 1. COMPOUND 1 has the following structure:

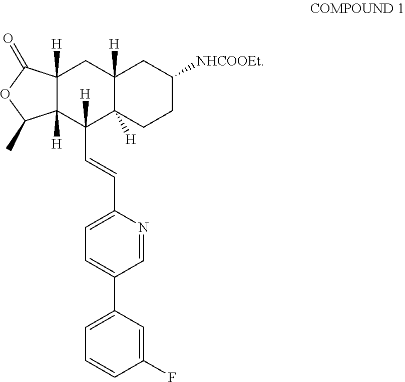

COMPOUND 1 is a potent and selective thrombin receptor antagonist, and is currently in development by Schering Corp. Co-pending U.S. patent application Ser. No. 10/705,282, herein incorporated by reference, discloses a variety of indications and combination formulations for thrombin receptor antagonists including COMPOUND 1. A preferred crystalline form of the bisulfate salt of COMPOUND 1 is disclosed in U.S. Pat. No. 7,235,567. U.S. patent application Ser. Nos. 11/771,571; 11/771,520; and 11,860,165 disclose capsule formulations, tablet formulations and lyophilized formulations (respectively) of Compound 1, and methods of treating various conditions by administering same.

The use of a small subset of thrombin receptor antagonists to treat a variety of conditions and diseases is disclosed in U.S. publication no. 04/0192753. The prevention of complications associated with cardiopulmonary bypass surgery by administration of a thrombin receptor antagonist is described in U.S. application Ser. No. 11/613,450. Substituted thrombin receptor antagonists are disclosed in U.S. Pat. Nos. 6,063,847; 6,326,380; and 6,645,987 and U.S. publication nos. 03/0203927; 04/0216437A1; 04/0152736; and 03/0216437. All of the herein cited references are incorporated in their entirety.

It would be beneficial to provide dosing regimens to achieve acceptable pharmacokinetic characteristics for COMPOUND 1. The invention seeks to provide these and other benefits, which will become apparent as the description progresses.

### SUMMARY OF THE INVENTION

In some embodiments, the present invention is directed to a method of treating acute coronary syndrome or peripheral vascular disease or of effecting secondary prevention in a person in need of such treating or effecting comprising administering to said person a loading dose of a pharmaceutical composition comprising Compound 1 and a pharmaceutically acceptable carrier, wherein said administering results in a mean plasma Cₘₐₓ of between about 238 and about 511 ng/mL at a Tₘₐₓ of between about 0.5 and about 1.5 hours. (Table 3)

In some embodiments, the loading dose comprises about 40 mg of Compound 1.

In some embodiments, administration of Compound 1 further results in an AUC_{(0-72 hr)} of between about 4745 and about 7508 ng-hr/mL.

Some embodiments further comprise the step of administering a maintenance dose of a pharmaceutical composition comprising Compound 1 and a pharmaceutically acceptable carrier, wherein said maintenance dose is administered once daily for a period equal to at least 5 times the mean terminal-phase half-life.

In some embodiments, the maintenance dose comprises about 1 mg of Compound 1 and the mean terminal-phase half-life is about 269 hours.

In some embodiments, the maintenance dose comprises about 3 mg of Compound 1 and the mean terminal-phase half-life is about 173 hours.

In some embodiments, the maintenance dose comprises between about 1 and about 3 mg of Compound 1 and the mean terminal-phase half-life is between about 173 hours and about 269 hours.

In some embodiments, the maintenance dose comprises about 5 mg of Compound 1 and the mean terminal-phase half-life is about 217 hours.

In further embodiments, the present invention is directed to a method of achieving a steady state plasma concentration of a thrombin receptor antagonist in a person in need of such plasma concentration comprising administering to the person a maintenance dose of a pharmaceutical composition comprising the thrombin receptor antagonist and a pharmaceutically acceptable carrier, wherein said maintenance dose is administered once daily (for a period equal to at least 4-5 times the mean terminal-phase half-life.)

In some embodiments, the thrombin receptor antagonist is selected from the group consisting of

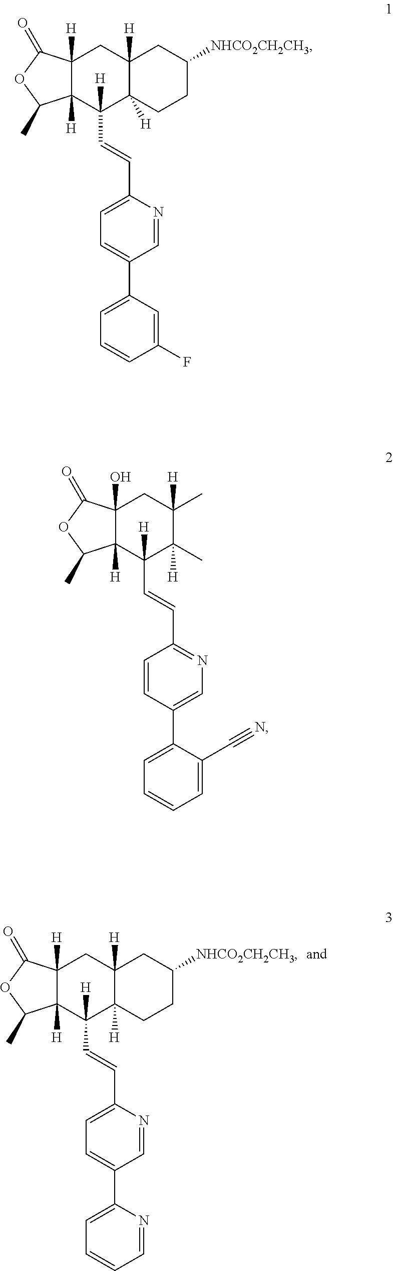

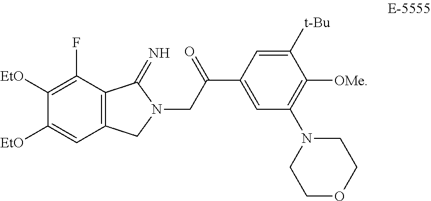

In some embodiments, the thrombin receptor antagonist is

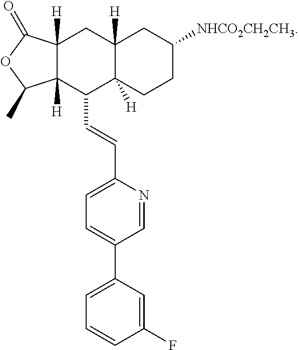

In yet further embodiments, the present invention is directed to a method of treating acute coronary syndrome or peripheral vascular disease or of effecting secondary prevention in a person in need of such treating or effecting comprising administering a maintenance dose of a thrombin receptor antagonist, wherein said maintenance dose is administered once daily (for a period equal to at least 4-5 times the mean terminal-phase half-life).

In some embodiments, the thrombin receptor antagonist is selected from the group consisting of

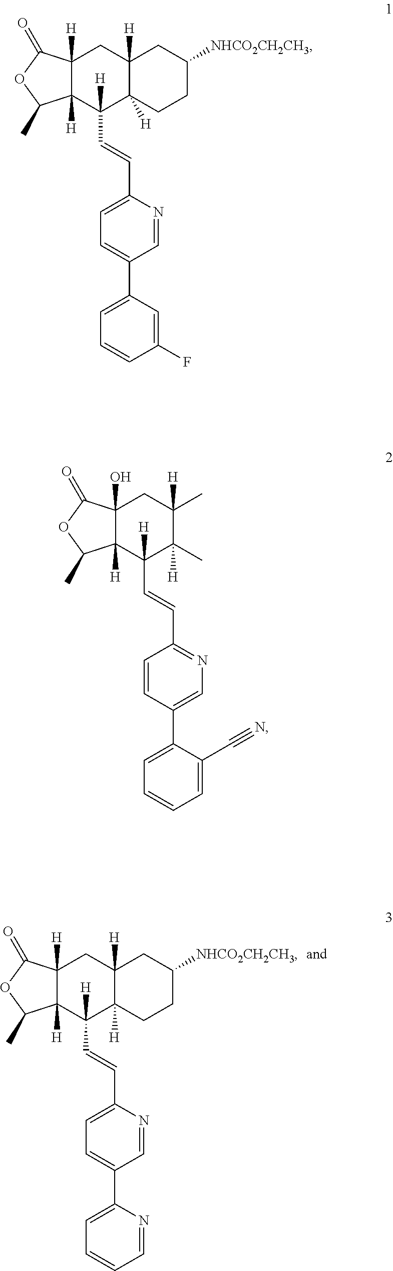

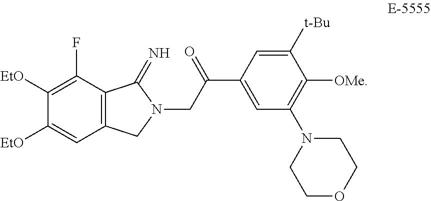

In some embodiments, the thrombin receptor antagonist is

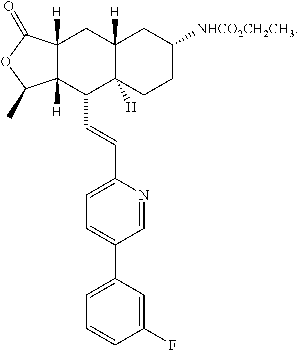

A further understanding of the invention will be had from the following description and claims.

### BRIEF DESCRIPTION OF THE DRAWINGS

FIG. 1 illustrates mean plasma COMPOUND 1 concentrations following single oral doses.

FIG. 2 illustrates individual (scatter) and mean (line) COMPOUND 1 exposure following a single dose of COMPOUND 1.

FIG. 3 illustrates mean plasma COMPOUND 1 concentrations following multiple oral doses.

FIG. 4 illustrates individual (scatter) and mean (line) COMPOUND 1 exposure following multiple oral doses.

FIG. 5 illustrates individual Plasma COMPOUND 1 Pre-Dose Concentration-Time Profiles.

### DESCRIPTION OF THE INVENTION

Schering Corp. is developing a thrombin receptor antagonist (“TRA”) for use in a variety of cardiovascular applications, including treatment of acute coronary syndrome and secondary prevention. The active pharmaceutical ingredient (“API”), COMPOUND 1, has completed phase I and II clinical trials. Dosing regimens being considered for commercialization include potential loading doses of about 10, 20 and 40 mg and maintenance doses of about 0.5, 1, 2.5 and 5 mg, in formulations for oral administration. Based on clinical data, it appears that a maintenance dose of between 0.25 and 5 mg will safely achieve therapeutically effective blood levels of COMPOUND 1 in a patient in the desired time frame. A loading dose of 40 mg and a maintenance dose of 2.5 mg are in phase III clinical trials. The development of formulations of suitable pharmaceutical characteristics is a necessary step in the commercialization of this thrombin receptor antagonist.

“Acute coronary syndrome” includes any group of clinical symptoms compatible with acute myocardial ischemia. Acute myocardial ischemia is chest pain due to insufficient blood supply to the heart muscle that results from coronary artery disease (also called coronary heart disease). Acute coronary syndrome thus covers the spectrum of clinical conditions ranging from unstable angina to non-Q-wave myocardial infarction and Q-wave myocardial infarction. Symptoms may include chest pain, shortness of breath, nausea, vomiting, diaphoresis (sweating), palpitations, anxiety or a sense of impending doom and a feeling of being acutely ill.

“Secondary prevention” refers to the treatment of patients who have already suffered a significant cardiovascular event, such as a heart attack or stroke, to prevent another future, potentially more serious, perhaps lethal, cardiovascular or cerebrovascular event.

Another cardiovascular condition for which thrombin receptor antagonists may be useful is peripheral arterial disease (“PAD”), also known as peripheral vascular disease (“PVD”), which occurs when cholesterol and scar tissue build up, forming plaque inside the arteries that narrows and clogs the arteries. The clogged arteries cause decreased blood flow to the legs, which can result in pain when walking, and eventually gangrene and amputation.

For the first-in-human, phase I studies, 0.25-, 1.0-, and 5.0-mg capsule formulations of COMPOUND 1 were developed using a simple dry blending process. The prototype formulations were developed based on the results of excipient compatibility studies, dissolution screening studies and content uniformity studies. These prototype formulations, labeled “A,” “B” and “C” are displayed in Table 1.

TABLE 1
Prototype Capsule Formulations

| | | Amount (mg) | | |
|---|---|---|---|---|
| | | Formulation | | |
| Ingredient | Function | A | B | C |
| COMPOUND 1 | TRA | 0.25 | 1.0 | 5.0 |
| (bisulfate) | | | | |
| Microcrystalline | Diluent | 140.75 | 140.0 | 136.0 |
| Cellulose | | | | |
| Crospovidone | Disintegrant | 7.5 | 7.5 | 7.5 |
| Magnesium Stearate | Lubricant | 1.5 | 1.5 | 1.5 |
| Capsule Fill Weight | | 150.0 | 150.0 | 150.0 |
| Capsule, No. 2 Blue | Capsule Shell | | | |
| Opaque Gelatin | | | | |

The present invention encompasses dosing regimens of solid formulations of any thrombin receptor antagonist. A variety of compounds have been demonstrated as displaying activity as thrombin receptor antagonists, many being himbacine analogs. As disclosed in U.S. publication no. 04/0152736, a subset of particularly preferred compounds of Formula I is as follows:

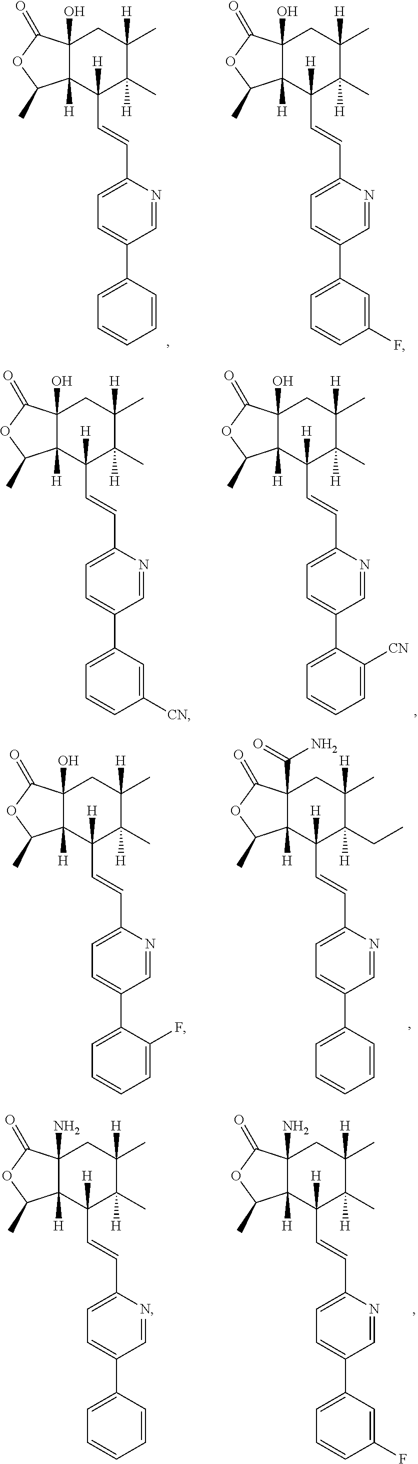

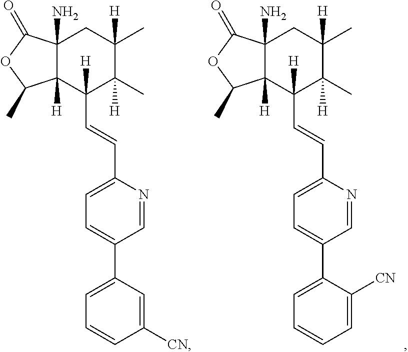

and the pharmaceutically acceptable isomers, salts, solvates and polymorphs thereof. U.S. publication no. 03/0216437 discloses a subset of thrombin receptor antagonists of Formula II which are both particularly active and selective. These compounds are as follows:

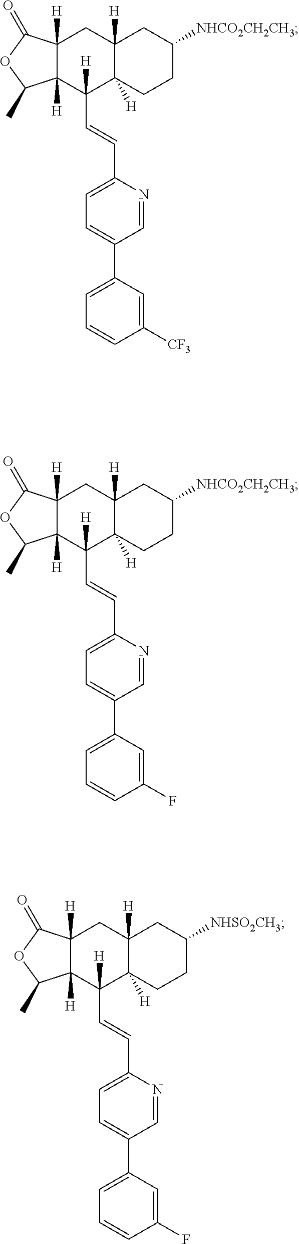

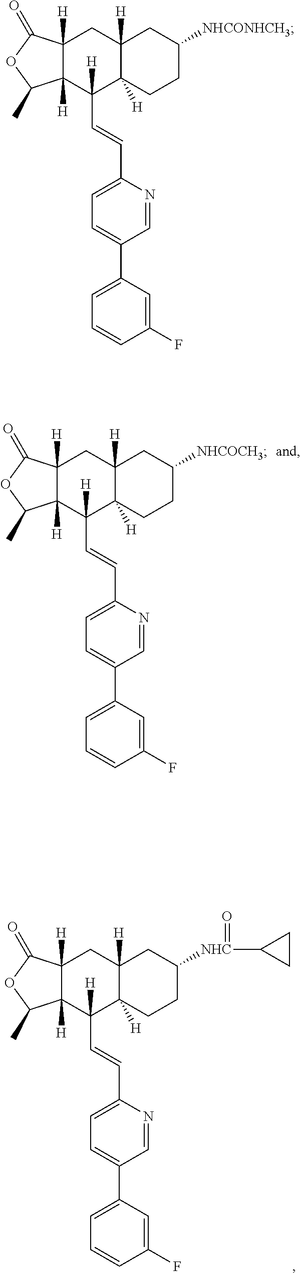

and the pharmaceutically acceptable isomers, salts, solvates and polymorphs thereof.

The following compounds are particularly favored based on their pharmacokinetics and pharmacodynamic characteristics:

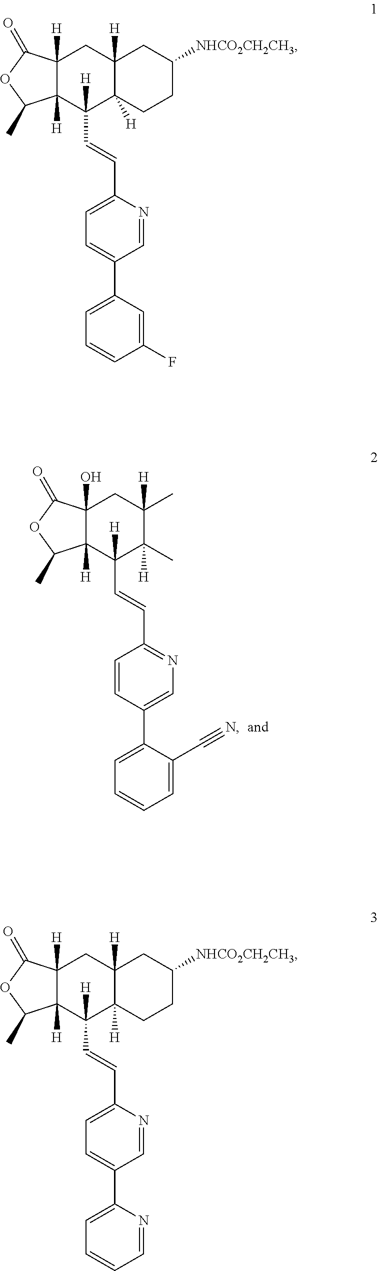

and the pharmaceutically acceptable isomers, salts, solvates and polymorphs thereof. The bisulfate salt of COMPOUND 1 is currently in development as a thrombin receptor antagonist by Schering Corp. Its synthesis is disclosed in U.S. publication no. 03/0216437, published Nov. 20, 2003, which publication also discloses Compound 3. Compound 2 is disclosed in U.S. Pat. No. 6,645,987.

Other compounds for use in the combinations of the present invention are disclosed in any of U.S. Pat. Nos. 6,063,847 and 6,326,380, U.S. Patent Publications 03/0203927, 03/0216437, 04/0192753 and 04/0176418, all of which are incorporated by reference in their entirety. Combinations that include one or more other agents that display activity as thrombin receptor antagonists are also within the scope of the present invention, including E5555 currently in development by Eisai:

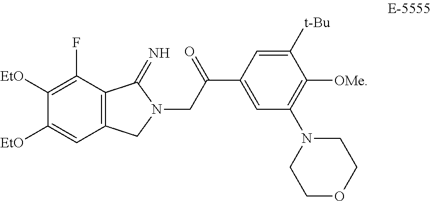

It will be understood that unless otherwise specified, the term “thrombin receptor antagonist” and any compounds identified as such, including COMPOUND 1, encompass any chemically stable and pharmaceutically acceptable free base, salt, isomer or solvate form thereof. The term “salt(s)”, as employed herein, denotes acidic salts formed with inorganic and/or organic acids. Pharmaceutically acceptable (i.e., non-toxic, physiologically acceptable) salts are preferred, although other salts are also useful. Salts of the compound of the above active agents may be formed, for example, by reacting the above active agents with an equivalent amount of acid or base in a medium such as one in which the salt precipitates or in an aqueous medium followed by lyophilization.

Phase I clinical studies were designed to evaluate the pharmacokinetics of COMPOUND 1 in healthy subjects after rising single and multiple doses. The study included a randomized, evaluator-blind, placebo-controlled, rising single dose (“RSD”) study and a rising multiple dose (“RSM”) study of orally administered COMPOUND 1 in healthy subjects.

In the single-dose study, 50 healthy males were enrolled into 6 sequential cohorts and randomized to receive in ascending dose manner placebo (n=2-3/group) or oral COMPOUND 1 (0.25, 1, 5, 10, 20 and 40 mg; n=5-6/group). In the multiple-dose study, 36 healthy subjects were enrolled into 3 sequential cohorts and randomized to receive in ascending-dose manner placebo (n=4/group) or oral COMPOUND 1(1, 3, and 5 mg OD in the morning for 28 days, n=8/group). A fourth cohort in the same study (n=12) was randomized to receive a single loading dose of COMPOUND 1, 10 mg (n=6) or 20 mg (n=6) on Day 1, followed by maintenance doses of 1 mg daily for 6 days. Measured and derived pharmacokinetic parameters were used to assess the pharmacokinetic profile. The designs of the RSD and RSM studies are summarized in Table 2. The doses were administered using appropriate combinations of the capsule formulations disclosed in Table 1.

TABLE 2

| | | Rising Multiple |
|---|---|---|
| | Rising Single Dose Study | Dose Study |
| Treatment | 0.25 mg, 1 mg, 5 mg, 10 mg, | 1 mg, 3 mg, 5 mg or |
| | 20 mg, 40 mg or placebo | placebo, once daily |
| | | for 28 days |
| Subjects | Healthy male subjects, | Healthy male |
| | n = 6/dose (2:1 randomization) | and female subjects; |
| | | n = 8/dose (2:1 |
| | | randomization) |
| Pharmacokinetic | 0-72 hr | 0-24 hr Day 1 |
| sample | extended to 64 days post-dose | 0-24 hr Day 28 |
| collection | for 20 mg and 40 mg groups | 0 hr (pre-dose) |
| | | Days 7, 14, 21, 26 |
| | | and 28 |

| | |
|---|---|
| Bioanalytical | LC-MS/MS (LLOQ = 0.1 ng/ml) |
| assay | |
| Pharmacokinetic | Model-independent methods |
| Analysis | |

Several observations were made from the data collected in the RSD Study. Table 3 displays observed mean Cₘₐₓ, Tₘₐₓ, and AUC_{(0-72 hr)} following administration of single oral dosages of COMPOUND 1 in doses of 0.25, 1, 3, 5, 10, 20 and 40 mg. For the 40 mg dose, the minimum and maximum individual Cₘₐₓ and corresponding Tₘₐₓ, as well as the minimum and maximum individual AUC_{(0-72 hr)} are also displayed. The data show that COMPOUND 1 exhibited rapid absorption following oral administration. Tₘₐₓ ranged from 0.5 to 2 hrs after dosing.

TABLE 3
Pharmacokinetic parameters following administration
of single oral dosages of COMPOUND 1.

| | | | |
|---|---|---|---|
| | | | AUC_{(0-72 hr)} |
| Dose (n) | Cₘₐₓ (ng/mL) | Tₘₐₓ (hr) | (ng · hr/mL) |

| | | | |
|---|---|---|---|
| 0.25 mg (6) | 2.23 (8) | 1.0 (0.5-1.5) | 33.7 (28) |
| 1 mg (6) | 9.29 (18) | 1.25 (0.5-1.5) | 142 (20) |
| 5 mg (6) | 51.4 (17) | 0.75 (0.5-1.5) | 740 (11) |
| 10 mg (6) | 99.2 (19) | 1.5 (1.0-2.0) | 1514 (10) |
| 20 mg (5) | 183 (19) | 1.5 (1.0-2.0) | 3346 (7) |
| 40 mg (5) | | | |
| Mean | 376 (28) | 1.5 (0.5-1.5) | 6028 (18) |
| Min | 238 | 0.5 | 4745 |
| Max | 511 | 1.5 | 7508 |

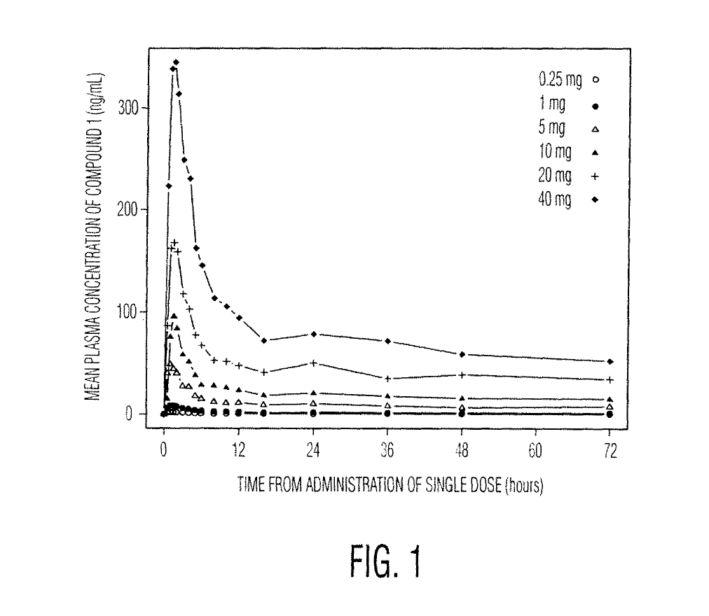

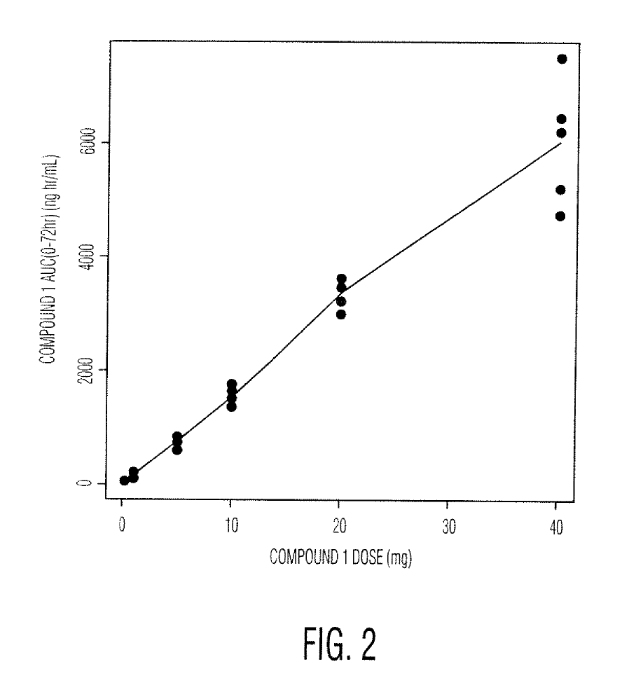

The pharmacokinetic profile of COMPOUND 1 was characterized by a fast distribution phase followed by a slow terminal elimination phase, as demonstrated in FIG. 1. Exposure to COMPOUND 1 was dose related at doses up to 40 mg, as shown in FIG. 2. To estimate the terminal-phase half-life (t½) of COMPOUND 1, samples were collected from subjects in the 20 mg and 40 mg dose groups for up to 64 days after dosing. The apparent terminal-phase half-life (t/½) in these subjects ranged from 126 to 269 hrs. Plasma concentrations were quantifiable for up to 53 days in subjects administered 20 mg and up to 62 days in subjects administered 40 mg. The variability in exposure to COMPOUND 1 was low; the coefficient of variation for AUC_{(0-72 h)} ranged from 7% to 28%.

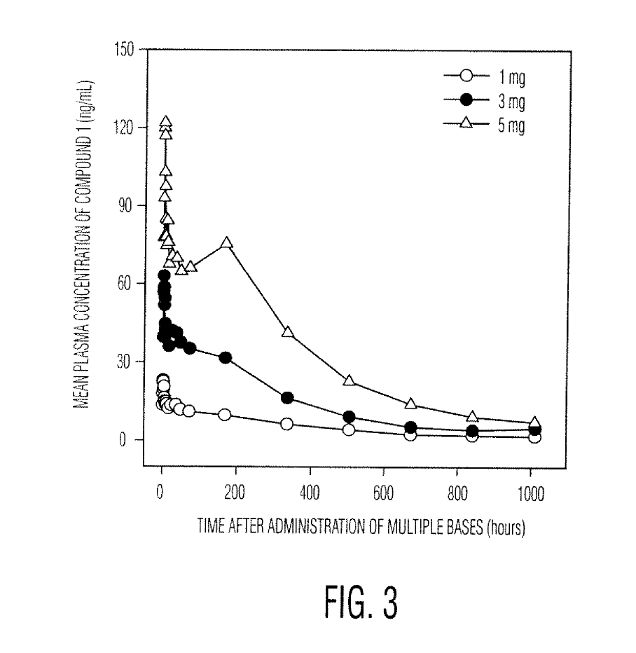

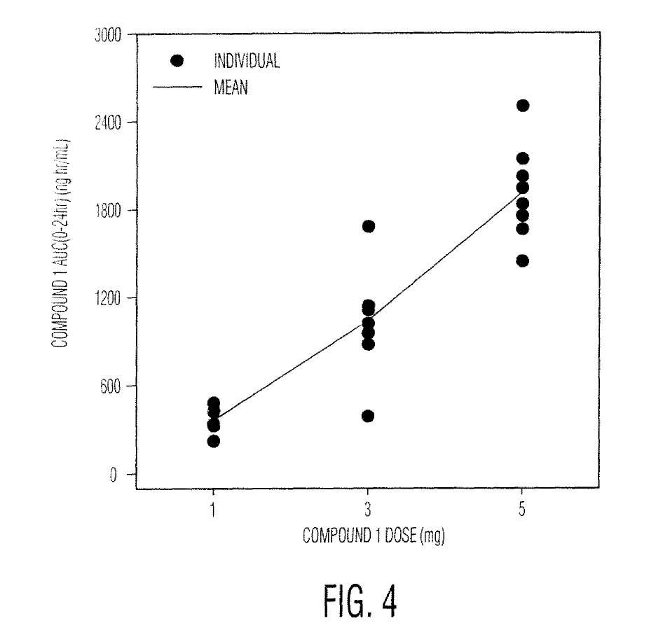

The rising multiple dose study resulted in a data set that supports its own set of observations. The rapid absorption and slow elimination following oral administration seen in the RSD study were also seen in the RMD study, as demonstrated by the data in Table 4 and FIG. 3. Median Tₘₐₓ values were observed from 1 to 1.5 hrs after dosing. Mean terminal-phase half-life (t½) ranged from 173 to 269 hrs. Also consistent with the results of the RSD study, it was found that exposure to COMPOUND 1 increased in a dose-related manner for all dose groups, as demonstrated in FIG. 4. Variability was low to moderate. The coefficient of variability (CV) for AUC_{(0-24 h)} ranged from 14% to 34%.

The RMD study also allowed evaluation of COMPOUND 1 accumulation. Mean accumulation index (R) values ranged from 4.72 to 6.37. The effective half-lives determined from R values ranged from 59.8 to 141 hrs.

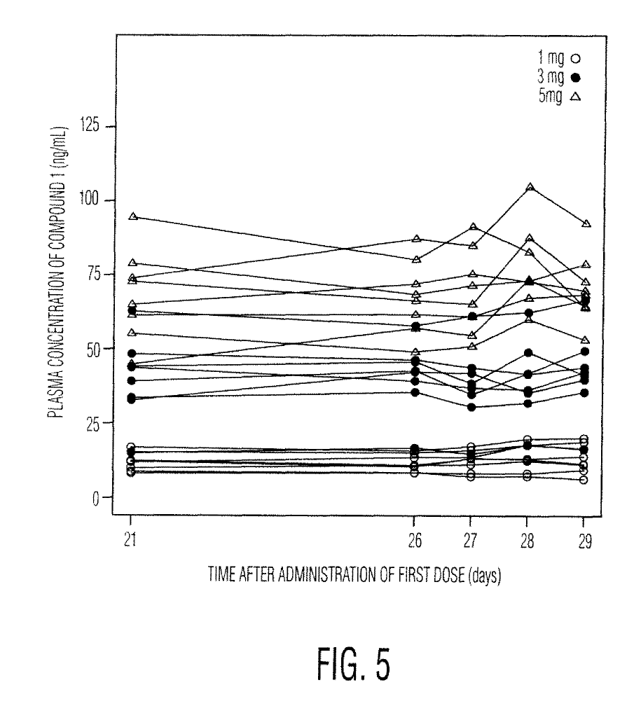

There was no consistent trend in the fluctuations of trough plasma COMPOUND 1 concentrations at the end of the dosing interval, as demonstrated in FIG. 5. Therefore, steady-state plasma concentrations were attained by Day 21 in all three dose groups, consistent with an effective half-life of about 60 to 141 hours.

TABLE 4
COMPOUND 1 pharmacokinetic parameters following single (Day 1) and multiple (Day 28) oral doses.

| | Cₘₐₓ | | Tₘₐₓ | | t_{1/2} | | AUC_{(0-24 hr)} | | | |
|---|---|---|---|---|---|---|---|---|---|---|
| | (ng/mL) | | (hr) | | (hr) | | (ng · hr/mL) | | R | |
| Dose | Mean | CV (%) | Median | Range | Mean | CV (%) | Mean | CV(%) | Mean | CV (%) |
| Single Dose: | Day 1 | | | | | | | | | |
| 1 mg (n = 8) | 9.78 | 31 | 1.00 | 0.5-4.0 | NA | NA | 58.5 | 24 | NA | NA |
| 3 mg (n = 8) | 38.2 | 25 | 1.00 | 0.5-1.0 | NA | NA | 225 | 15 | NA | NA |
| 5 mg (n = 8) | 56.4 | 25 | 1.00 | 0.5-2.0 | NA | NA | 315 | 21 | NA | NA |
| 10 mg (n = 6) | 85.4 | 24 | 1.00 | 1.0-2.0 | NA | NA | 592 | 14 | NA | NA |
| 20 mg (n = 6) | 188 | 32 | 1.50 | 1.0-4.0 | NA | NA | 1280 | 23 | NA | NA |
| Multiple Dose: | Day 28 | | | | | | | | | |
| 1 mg (n = 7) | 24.6 | 24 | 1.00 | 0.5-2.0 | 269 | 30 | 363 | 24 | 6.37 | 18 |
| 3 mg (n = 8) | 65.8 | 37 | 1.00 | 0.5-2.0 | 173 | 35 | 1040 | 34 | 4.72 | 33 |
| 5 mg (n = 8) | 131 | 18 | 1.00 | 1.0-2.0 | 217 | 22 | 1910 | 17 | 6.22 | 20 |

In conclusion, both the single and multiple oral dosing studies, COMPOUND 1 was rapidly absorbed and distributed, in a dose-related manner. Maximal plasma levels were attained generally within 60-90 minutes across all doses in both studies. The exposure to COMPOUND 1 was dose-related, and the variability of exposure was low in both studies. Elimination of COMPOUND 1 was slow in both studies (mean t½=173-269 hr). Accumulation (5- to 7-fold) was observed in the multiple dosing study, and the observed steady state plasma concentrations of COMPOUND 1 were attained by day 21. The pharmacokinetic profile of COMPOUND 1 observed in this study supports once-daily oral dosing.

The data generated in the phase I trials suggest methods of treating acute coronary syndrome or peripheral vascular disease or of effecting secondary prevention by administering the single doses listed in Table 3 (i.e., 0.25, 1, 5, 10, 20 and 40 mg) to achieve each respective Cₘₐₓ at the Tₘₐₓ, and further to achieve the respective AUC_{(0-72 hr)}, as reflected in Table 3. The data further suggest methods of treating acute coronary syndrome or peripheral vascular disease or of effecting secondary prevention by a single administration of each of the loading doses listed in Table 4 (i.e., 1, 3, 5, 10, and 20 mg) followed by daily administration of each of the maintenance doses (i.e., 1, 3 and 5) to achieve after 28 days each respective Cₘₐₓ at the Tₘₐₓ, and further to achieve the respective AUC_{(0-24 hr)}, as reflected in Table 4.

Dosing regimens for the thrombin receptor antagonists referred to herein will include a single administration of a loading dose, followed by daily administrations of a maintenance dose. Since platelet inhibition data have suggested that 40 mg is a preferred dose for the loading dose, dosing regimens that provide the pharmacokinetic results of such a loading dose are also preferred. Referring to Table 3, said administering results in a plasma concentration range Cₘₐₓ of between about 238 and about 511 ng/mL (with a mean of about 376 ng/mL) at a Tₘₐₓ of between about 0.5 and about 1.5 hours, and an exposure range AUC_{(0-72 hr)} of between about 4745 and about 7508 ng-hr/mL (with a mean of about 6028 ng-hr/mL). Thus, any dosing regimen of a loading dose that results in these pharmacokinetic parameters is also within the scope of the present invention.

Since platelet inhibition data suggest that a maintenance dose of between about 1 mg and about 3 mg is preferred, (about 2.5 mg), the pharmacokinetic characteristics of such a maintenance dose are also preferred. The mean terminal-phase half-life is an example of a particularly useful pharmacokinetic characteristic. Dosing regimens that provide for administration of the maintenance dose for a period equal to at least 4-5 times the mean terminal-phase half-life and the effective half-life can result in steady state pharmacokinetics, and are thus preferred. The criterion for achieving “steady state” will be understood to be steady or increasing (i.e., not decreasing) pre-dose trough values for Compound 1. Thus, maintenance doses that have a mean terminal-phase half-life of between about 269 and about 173 hours (see Table 4) or the effective half-life of about 60 to about 141 hours are preferred, and dosing regimens that include once daily dosing of a maintenance dose for a period of at least 4-5 times these mean terminal-phase half-lives and effective half-lives are also preferred.

Although the clinical data presented herein were generated by dosing patients with capsule formulations, the dosing regimens and pharmaceutical formulations of the present invention are not limited to those involving capsule formulations. Tablet formulations are the preferred pharmaceutical formulations, and dosing regimens that relate to administration of tablet formulations are also preferred. For the loading dose, lyophilized formulations represent alternate preferred embodiments.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A method of treating acute coronary syndrome or peripheral vascular disease or of effecting secondary prevention in a person in need of such treating or effecting comprising administering to said person a loading dose of a pharmaceutical composition comprising Compound 1 and a pharmaceutically acceptable carrier, wherein said administering results in a mean plasma Cₘₐₓ of between about 238 and about 511 ng/mL at a Tₘₐₓ of between about 0.5 and about 1.5 hours.

2. The method according to claim 1 wherein said loading dose comprises about 40 mg of Compound 1.

3. The method according to claim 1, wherein said administering further results in an AUC_{(0-72 hr)} of between about 4745 and about 7508 ng-hr/mL.

4. The method according to claim 1 further comprising the step of administering a maintenance dose of a pharmaceutical composition comprising Compound 1 and a pharmaceutically acceptable carrier, wherein said maintenance dose is administered once daily for a period equal to at least 4-5 times the mean terminal-phase half-life or effective half-life.

5. The method according to claim 4, wherein the maintenance dose comprises about 1 mg of Compound 1 and the mean terminal-phase half-life is about 269 hours.

6. The method according to claim 4, wherein the maintenance dose comprises about 3 mg of Compound 1 and the mean terminal-phase half-life is about 173 hours.

7. The method according to claim 4, wherein the maintenance dose comprises between about 1 and about 3 mg of Compound 1 and the mean terminal-phase half-life is between about 269 and about 173 hours or effective half life of about 60 to about 141 hours.

8. The method according to claim 4, wherein the maintenance dose comprises about 5 mg of Compound 1 and the mean terminal-phase half-life is about 217 hours.

9. A method of achieving a steady state plasma concentration of a thrombin receptor antagonist in a person in need of such plasma concentration comprising administering to the person a maintenance dose of a pharmaceutical composition comprising the thrombin receptor antagonist and a pharmaceutically acceptable carrier, wherein said maintenance dose is administered once daily for a period equal to at least 4 to 5 times the mean terminal-phase half-life or effective half-life.

10. The method according to claim 9 wherein said thrombin receptor antagonist is selected from the group consisting of

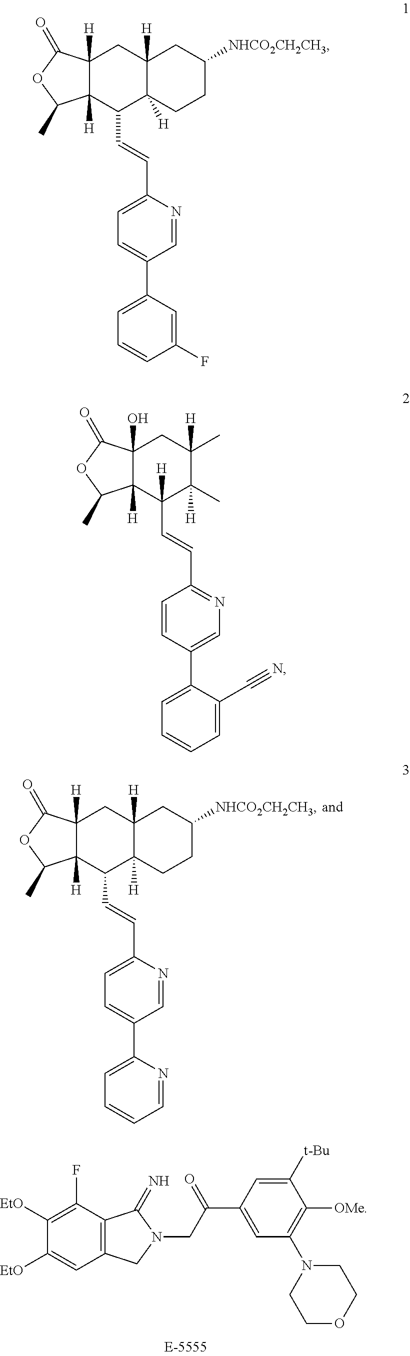

11. The method according to claim 9 wherein said thrombin receptor antagonist is

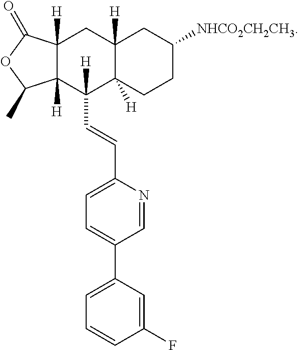

12. A method of treating acute coronary syndrome or peripheral vascular disease or of effecting secondary prevention in a person in need of such treating or effecting comprising administering a maintenance dose of a thrombin receptor antagonist, wherein said maintenance dose is administered once daily for a period equal to at least 5 times the mean terminal-phase half-life.

13. The method according to claim 12 wherein said thrombin receptor antagonist is selected from the group consisting of

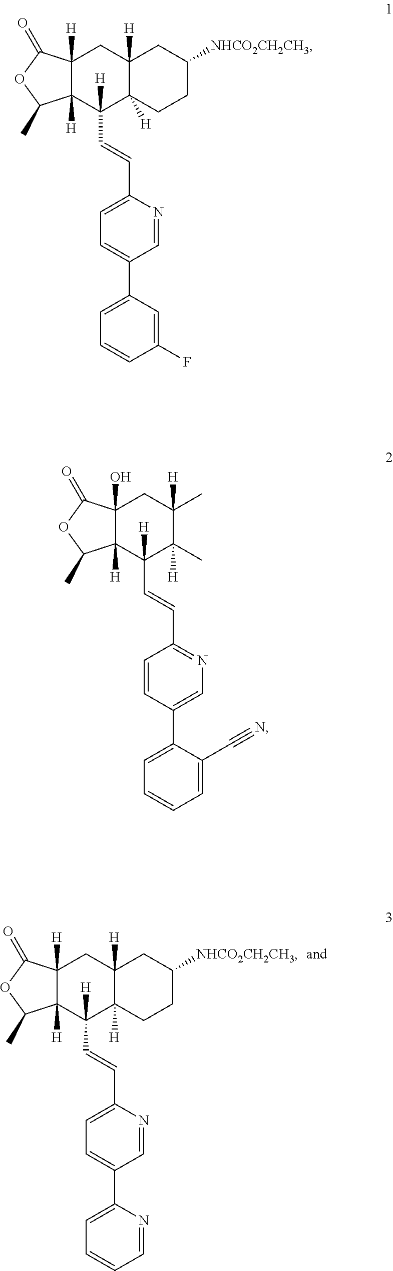

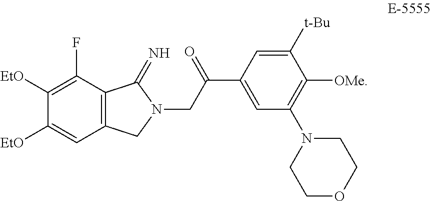

14. The method according to claim 12 wherein said thrombin receptor antagonist is [chemical structure]
